(19) United States
(12) Patent Application Publication
(10) Pub. No.: US 2005/0255174 A1
(43) Pub. Date: Nov. 17, 2005

(54) Process and appratus for use in preparing an aqueous magnesium bicarbonate solution

(76) Inventors: Arthur Shelley, Taren Point (AU); Dennis Shelley, Taren Point (AU); Russell John Beckett, Red Hill (AU)

Correspondence Address: SEYFARTH SHAW LLP, 55 EAST MONROE STREET, SUITE 4200, CHICAGO, IL, 60603-5803, US

(21) Appl. No.: 10/511,008

(22) PCT Filed: Apr. 7, 2003
(86) PCT No.: PCT/AU03/00417

§ 371 Date: May 20, 2005

(30) Foreign Application Priority Data
Apr. 5, 2002 (AU) PS 1568
Aug. 19, 2002 (AU) 2002950860

(51) Int. Cl. A61K 33/10

(52) U.S. Cl. 424/686

(57) ABSTRACT

A method and an apparatus for preparing a substantially clear aqueous solution containing magnesium bicarbonate are disclosed. One method of preparing a substantially clear aqueous solution containing magnesium bicarbonate, includes contacting, with species resulting from the dissolution of carbon dioxide in water, an aqueous suspension of magnesium carbonate, at suitable conditions of pressure and temperature, so as to obtain an aqueous solution of magnesium bicarbonate and controlling the pH of the said solution so that, after reaction of the said species with the magnesium carbonate, the final pH falls within a range of from about 8.0 to about 8.8. One apparatus suitable for preparing an aqueous solution of magnesium bicarbonate, comprises means for contacting, with species resulting from the dissolution of carbon dioxide in water, a suspension of powdered magnesium carbonate in water so as to form an aqueous solution of magnesium bicarbonate and means for controlling the pH of the solution between about 7 and about 9 by adjusting the amount of at least one of the said species and said powdered magnesium carbonate that is contacted with the other.

### TECHNICAL FIELD

The present invention relates to the preparation of an aqueous magnesium bicarbonate solution. More particularly, the invention relates to processes and apparatus for use in the production of an aqueous magnesium bicarbonate solution suitable for oral ingestion,

### BACKGROUND OF THE INVENTION

The consumption or administration of sodium and potassium bicarbonates has been correlated to the neutralisation of acid in the human body. Amongst other effects, the neutralisation of acid with bicarbonates (potassium bicarbonate in particular) decreases bone resorption, increases the rate of bone formation and stops the decline in muscle and tissue wasting that occurs with ageing and degenerative diseases (see Sebastian et al, 1994 N Eng J Med 330 (25): 1776-81 and Frassetto et al 1997 J Clin Endocrin Metab 82 (1): 254-259).

However, both sodium and potassium bicarbonates are known to be toxic to some people at moderate to high dosage rates when consumed for a prolonged period. Thus, there is a need for a bicarbonate in a safe form.

There is also a need for a bicarbonate suitable for the prevention and treatment of diseases, in humans and other mammals, where the diseases have high carbon dioxide concentrations and/or high acid concentrations in the disease pathogenesis.

In Patent Application PCT/AU/98/00168 and in Australia Patent No 735 435, a process for the manufacture of a mildly alkaline magnesium bicarbonate solution from a basic magnesium carbonate powder dispersed in water, using carbon dioxide absorbed into the water from the gaseous state, is described. In that process, dissolved carbon dioxide reacts with the magnesium carbonate either while it is still in its solid form as a suspension in water or upon dissolution thereof in water, to form magnesium bicarbonate:

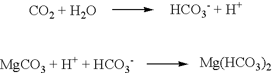

The time required for the above chemical reactions to run to completion to ensure the clarity or clearness of the final solution was stated in Patent Application PCT/AU/98/00168 and in Australia Patent No 735435 to be not less than 6 hours, typically from 6 hours to seven days, more typically from 24 hours to 72 hours.

It has been found by the applicant to be undesirable to bottle magnesium bicarbonate for oral administration, manufactured from magnesium carbonate by the dissolution thereof with carbon dioxide before it has been stabilised.

On the one hand, it is desirable for the solution to be clear. Because any undissolved magnesium carbonate does not dissolve easily after the solution has been bottled, it is desirable to dissolve all magnesium carbonate powder before bottling the solution, or to bottle the solution when there is no more than a slight haze visible in it, particularly when the pH of the solution is not close to or at the maximum of the aforementioned range of 7.5 to 8.9.

On the other hand, it is desirable for the magnesium bicarbonate solution to have a pH higher than the physiological pH of a mammal, preferably as high as possible within the aforesaid range of 7.5 to 8.9, consistent with the objective of maintaining a clear solution, without the higher pH causing magnesium carbonate to be either precipitated from the solution or not being dissolved or converted into magnesium bicarbonate in the first place.

There accordingly exists a need for a process for the manufacture of a magnesium bicarbonate containing solution that has a decreased reaction time under temperature conditions which are favourable for the rapid dissolution of carbon dioxide in water, without the solution having a pH which is too low.

There also exists a need for a process for the bottling of a magnesium bicarbonate containing solution that will be substantially clear or that will become substantially clear within a relatively short period after bottling, which period may be from one to eight hours after bottling.

### OBJECT OF THE INVENTION

It is an object of the present invention to overcome or substantially ameliorate at least one of the above disadvantages.

### SUMMARY OF THE INVENTION

According to a first aspect of the present invention, there is provided a method of preparing a substantially clear aqueous solution containing magnesium bicarbonate, including the steps of:
contacting, with species resulting from the dissolution of carbon dioxide in water, an aqueous suspension of magnesium carbonate, at suitable conditions of pressure and temperature, so as to obtain an aqueous solution of magnesium bicarbonate; and
controlling the pH of the said solution so that, after reaction of the said species with the magnesium carbonate, the final pH falls within a range of from about 8.0 to about 8.8.

According to a second aspect of the present invention, there is provided a method of preparing a substantially clear aqueous solution containing magnesium bicarbonate, including the step of contacting, with species resulting from the dissolution of carbon dioxide in water, an aqueous suspension of magnesium carbonate, at suitable conditions of pressure and temperature, so as to obtain an aqueous solution of magnesium bicarbonate and magnesium carbonate, said aqueous solution having a pH, after reaction of the said species with the magnesium carbonate, within a range of from about 8.0 to about 8.8.

The process according to the second aspect of the invention may conveniently therefore also include a step in which the pH is controlled.

The pH may be controlled by decreasing or increasing the pH of the aqueous suspension of magnesium carbonate and of the resulting solution by dissolving in the suspension a larger or a smaller amount of carbon dioxide. Alternatively, the pH may be controlled by increasing or decreasing the amount of magnesium carbonate introduced into the suspension. As another alternative, the pH may be controlled by introducing into the solution protons or a substance which has an effect on the pH.

According to a third aspect of the present invention, there is provided a method of preparing a substantially clear aqueous solution containing magnesium bicarbonate, including the steps of:
contacting, with species resulting from the dissolution of carbon dioxide in water, an aqueous suspension of magnesium carbonate, at suitable conditions of pressure and temperature, so as to obtain an aqueous solution of magnesium bicarbonate at a pH, after reaction of the said species with the magnesium carbonate, falling within a range of from about 8.0 to about 8.8; and
agitating the suspension so as to keep in suspension a major portion of the magnesium carbonate until substantially all of the said magnesium carbonate has been converted to dissolved magnesium bicarbonate.

The major portion of the magnesium carbonate may be any proportion from about 30% to about 100% of the magnesium carbonate forming part of the said suspension.

The method according to the second aspect of the invention may conveniently also include a step in which the pH is controlled. This may be achieved by either decreasing or increasing the pH of the aqueous suspension of magnesium carbonate and of the resulting solution by dissolving in the suspension a larger or a smaller amount of carbon dioxide.

Alternatively, the pH may be controlled by increasing or decreasing the amount of magnesium carbonate introduced into the suspension. As another alternative, the pH may be controlled by introducing into the solution protons or a substance which has an effect on the pH.

The pH of the aqueous suspension of magnesium carbonate may advantageously be controlled to be above about 8, ideally between about 8.2 and about 8.8.

The method according to these aspects of the invention may include the step of controlling the pressure at an absolute pressure of from about 50 kPa to about 500 kPa.

The aqueous suspension of magnesium carbonate preferably contains from about 50 mg to about 200 milligrams of magnesium per litre of water, more particularly from about 90 mg per litre to 150 mg per litre, even more particularly from about 100 mg per litre to about 140 mg per litre. The aqueous solution of magnesium bicarbonate obtained may have a concentration of bicarbonate anions from about 200 mg per litre to about 1000 mg per litre, more particularly from about 400 mg per litre to 800 mg per litre, even more particularly from about 500 mg per litre to 700 mg per litre.

By carefully following the teachings of the present invention, the aqueous solution of magnesium bicarbonate may be produced in commercial quantities in less than about 6 hours.

The species resulting from the dissolution of carbon dioxide in water may include protons and bicarbonate ions. Alternatively or additionally, it may include carbonic acid.

The magnesium carbonate is preferably contacted with a stoichiometric quantity of species resulting from the dissolution of carbon dioxide in water, or with a quantity of such species which exceeds a stoichiometric quantity by from about 0% to about 20%, preferably by no more than about 10%, more preferably, by no more than about 1% to about 5%.

The temperature of the aqueous suspension of magnesium carbonate may also be controlled. The temperature may be controlled between about 0° C. and about 25° C.

It is to be understood that at lower temperatures, the rate of dissolution of carbon dioxide in water is faster than at higher temperatures. However, the rate of the reaction of magnesium carbonate with the species resulting from the dissolution of carbon dioxide in water, to form magnesium bicarbonate, is lower at lower temperature. Thus, the temperature of the aqueous suspension of magnesium carbonate water may be controlled at such a level as to achieve a desired absorption of carbon dioxide into the suspension consistent with a desired rate of reaction between magnesium carbonate and the species formed as a result of the dissolution of carbon dioxide in the suspension. Preferably, the temperature is maintained below about 25° C. in the range between about 1° C. and about 20° C., more preferably below about 18° C. in the range between about 5° C. and about 18° C., even more preferably in the range between about 10° C. and about 18° C., ideally in the range of from about 12° C. to about 18° C., even more ideally between about 12° C. and about 15° C.

The contacting step of either the first or the second aspect of the invention may be conducted either continuously or batchwise.

The method in accordance with the first and second aspects of the invention may conveniently also include the additional step of bottling the said aqueous solution of magnesium bicarbonate, preferably under an atmosphere containing carbon dioxide, preferably at an absolute pressure of from about 50 kPa to about 500 kPa. Alternatively, the solution may be bottled under air or nitrogen, at a pressure from about 50 kPa to about 500 kPa.

The bottling step is preferably carried out when the magnesium bicarbonate solution is clear, or, in order to reduce processing time, whilst the turbidity of the magnesium bicarbonate solution is no more than a slight haze and the pH falls within the range of about pH 7.0 to pH 9.0, usually between about 7.5 to 9.0, more usually between 8.0 and 9.0 or pH 8.1 to pH 8.9 ideally from about pH 7.8 to pH 8.8, more ideally from about pH 8.0 to pH 8.6. It has been found that if the turbidity is low enough, the solution will clear up after bottling if the pH falls within this range, but when the turbidity is too high, the solution either does not clear up after bottling or the end pH, after complete reaction of said species with the magnesium carbonate, is too high. If the pH value exceeds pH 9.0, the taste of the magnesium bicarbonate solution becomes unacceptable as it tends to be too caustic. Above pH 9.0 carbonate floccules may reappear in the solution. If the pH value falls below pH 7.0, the medical value of the magnesium bicarbonate solution is largely negated.

In order to prevent the presence of unwanted foreign matter in the magnesium bicarbonate solution, the method according to the first, second and third aspects of the invention may include the step of passing any one or more of the suspension, the solution and the water to be used for the preparation of the aqueous magnesium carbonate suspension through a filter having a maximum pore size of about 10 microns, preferably having a maximum pore size of about 5 microns, more preferably having a maximum pore size of about 2 microns.

According to a fourth aspect of the present invention, there is provided an apparatus suitable for preparing an aqueous solution of magnesium bicarbonate, comprising:
means for contacting, with species resulting from the dissolution of carbon dioxide in water, a suspension of powdered magnesium carbonate in water so as to form an aqueous solution of magnesium bicarbonate; and
means for controlling the pH of the solution between about 7 and about 9 usually between about 7.5 to 9.0, more usually between 8.0 and 9.0 or 8.1 to 8.9 by adjusting the amount of at least one of the said species and the said powdered magnesium carbonate that is contacted with the other.

The apparatus according to the third aspect of the invention may also comprise means for keeping in suspension a major portion of said powdered magnesium carbonate.

According to a fifth aspect of the present invention, there is provided an apparatus suitable for preparing an aqueous solution of magnesium bicarbonate, comprising:
means for contacting, with species resulting from the dissolution of carbon dioxide in water, a suspension of powdered magnesium carbonate in water so as to form an aqueous solution of magnesium bicarbonate; and
means for keeping in suspension a major portion of said powdered magnesium carbonate until substantially all of the suspended magnesium carbonate has been converted to dissolved magnesium bicarbonate.

The apparatus according to the fifth aspect of the invention may also comprise means for controlling the pH of the solution between about 7 and about 9 usually between about 7.5 to 9.0, more usually between 8.0 and 9.0 or 8.1 to 8.9. This may be achieved by means adapted to adjust the amount of at least one of the said species and the said powdered magnesium carbonate that is present in the apparatus.

The apparatus according to the fourth and fifth aspects of the invention may also comprise means for bottling the solution of magnesium bicarbonate.

The means for contacting may comprise means, such as a tank of sufficient size, for containing the suspension and the species resulting from the dissolution of carbon dioxide in water. In a batch process, a tank large enough to contain all of the magnesium bicarbonate solution required for a production run in which a certain number of bottles are filled with the solution, may be provided. In the case of a continuous process, a smaller container may be sufficient for the contacting step, although a suitable reactor and/or clarifier or other similar but suitable pieces of equipment may be provided to ensure that the reaction between the magnesium carbonate and the said species is permitted to run to substantial completion.

The means for contacting with magnesium carbonate the species resulting from the dissolution of carbon dioxide in water may also comprise means for dissolving carbon dioxide in the water or in the said suspension so as to form a solution containing the said species.

The means for dissolving carbon dioxide may comprise a sparger comprising a fine mesh such as a stocking like mesh, or another suitable device capable of breaking up the carbon dioxide into small bubbles. The sparger may conveniently comprise a porous separation means adapted to separate pressurised carbon dioxide from water into which the carbon dioxide is to be dissolved, the pores of the separation means extending from a gas side thereof to a water side thereof, in use, and to break the carbon dioxide up into a plurality of small bubbles, for increasing the intimacy of contact between the carbon dioxide and the water.

The means for dissolving carbon dioxide may comprise, in addition to or as an alternative to the aforementioned sparger means, a tube and a helical baffle locatable within the tube, means for disposing the tube in a substantially vertical position, means for introducing carbon dioxide into a first end of the tube which, in use, is located below a second end of the tube, and means for introducing water or the said aqueous suspension of magnesium carbonate at either of the first end or the second end thereof, whereby carbon dioxide is allowed to bubble through the tube and to be intimately contacted with the water or said aqueous suspension of magnesium carbonate.

The apparatus may further comprise means for bottling the magnesium bicarbonate solution. The means may be or may comprise means for bottling the solution with air, nitrogen or carbon dioxide under a pressure from about 120 kPa to about 500 kPa.

According to a sixth aspect of the present invention, there is provided a method of preparing a substantially clear aqueous solution containing magnesium bicarbonate, including the steps of:
contacting, with species resulting from the dissolution of carbon dioxide in water, an aqueous suspension of magnesium carbonate, at suitable conditions of pressure and temperature, so as to obtain an aqueous solution of magnesium bicarbonate at a pH, after reaction of the said species with the magnesium carbonate, falling within a range of from about 8.0 to about 8.8;
controlling the pH of the said solution within the said range until substantially all of the said magnesium carbonate has been converted to dissolved magnesium bicarbonate; and
bottling the said magnesium bicarbonate solution.

According to a seventh aspect of the present invention, there is provided a method of preparing a substantially clear aqueous solution containing magnesium bicarbonate, including the steps of:
contacting, with species resulting from the dissolution of carbon dioxide in water, an aqueous suspension of magnesium carbonate, at suitable conditions of pressure and temperature, so as to obtain an aqueous solution of magnesium bicarbonate at a pH, after reaction of the said species with the magnesium carbonate, falling within a range of from about 8.0 to about 8.8;
agitating the suspension so as to keep in suspension a major portion of the magnesium carbonate until substantially all of the said magnesium carbonate has been converted to dissolved magnesium bicarbonate; and
bottling the said aqueous magnesium bicarbonate solution.

According to an eighth aspect of the present invention, there is provided an apparatus suitable for preparing an aqueous solution of magnesium bicarbonate, comprising:
means for contacting, with species resulting from the dissolution of carbon dioxide in water, a suspension of powdered magnesium carbonate in water so as to form an aqueous solution of magnesium bicarbonate;
means for controlling the pH of the solution between about 7 and about 9 usually between about 7.5 to 9.0, more usually between 8.0 and 9.0 or 8.1 to 8.9 by adjusting the amount of at least one of the said species and the said powdered magnesium carbonate that is contacted with the other; and
means for bottling the said solution of magnesium bicarbonate.

According to a ninth aspect of the present invention, there is provided an apparatus suitable for preparing an aqueous solution of magnesium bicarbonate, comprising:
means for contacting, with species resulting from the dissolution of carbon dioxide in water, a suspension of powdered magnesium carbonate in water so as to form an aqueous solution of magnesium bicarbonate;
means for keeping in suspension a major portion of said powdered magnesium carbonate until substantially all of the suspended magnesium carbonate has been converted to dissolved magnesium bicarbonate; and
means for bottling said solution of magnesium bicarbonate.

According to a tenth aspect of the present invention, there is provided a process for the manufacture of an aqueous solution comprising dissolved magnesium bicarbonate and dissolved magnesium carbonate, wherein the process includes the step of contacting an aqueous suspension of a source of magnesium cations with such an amount of a species resulting from the dissolution of carbon dioxide in water and having an initial pH below about 7 as is sufficient to result in the said aqueous solution comprising dissolved magnesium bicarbonate and dissolved magnesium carbonate having a pH of from about 7.5 to about 8.9 or 9.0, more usually between 8.0 and 9.0 or 8.1 to 8.9 and to be substantially clear.

The process according to the tenth aspect of the present invention may include a step in which the aqueous suspension of a source of magnesium cations is prepared. This step may include the suspension of a suitably pulverised or particulate solid compound of magnesium in water.

The process according to the tenth aspect of the present invention may also include a step in which the species resulting from the dissolution of carbon dioxide in water is prepared so as to result in a carbonated solution having a pH below 7, preferably well below 7. The pH of the carbonated solution may be controlled by the dissolution of such an amount of carbon dioxide as would result in the desired pH.

The species resulting from the dissolution of carbon dioxide in water may include protons and bicarbonate ions. Alternatively or additionally, it may include carbonic acid.

The process may include the step of contacting an aqueous suspension of a source of magnesium cations with carbon dioxide gas and mixing the contacted aqueous suspension. The contacted and mixed suspension may be recontacted with carbon dioxide gas one or more times. In one form of the invention, a flowing aqueous suspension of a source of magnesium cations is contacted with bubbles of carbon dioxide gas and the contacted aqueous suspension is subsequently mixed. In one particular form of the invention, a flowing aqueous suspension of a source of magnesium cations is contacted with bubbles of carbon dioxide gas in a contacting vessel which is linked to a mixing vessel and the contacted aqueous suspension is subsequently transferred to the mixing vessel where it is mixed and then recirculated to the contacting vessel. This process is repeated until the pH of the aqueous solution is between about 7.5 to 9.0, more usually between 8.0 and 9.0, 8.1 to 8.9 and even more usually between about 8.2 and 8.8 and thereafter the solution is mixed and optionally recirculated (without contacting the solution during the recirculation step with carbon dioxide) until the source of magnum cations has substantially dissolved to form an aqueous solution of magnesium bicarbonate and magnesium carbonate. The solution may be thereafter sterilised and bottled.

According to an eleventh aspect of the present invention, there is provided a process for the manufacture of an aqueous solution comprising dissolved magnesium bicarbonate or dissolved magnesium bicarbonate and dissolved magnesium carbonate, wherein the process includes the step of: (i) contacting an aqueous suspension of a source of magnesium cations with bubbles of carbon dioxide gas, (ii) agitating the contacted aqueous suspension, (iii) recontacting the agitated contacted aqueous suspension with bubbles of carbon dioxide gas, and (iv) repeating steps (i) to (iii) as required, to provide a pH in the aqueous solution of from about 7.5 to about 9.

The invention also extends to the product of the process according to the invention.

The aqueous solution may be substantially clear or may be slightly turbid. Usually a slightly turbid solution will become clear when it is placed in a closed container such as when it is bottled and thereafter allowed to stand.

The aqueous solution comprising dissolved magnesium bicarbonate or dissolved magnesium bicarbonate and dissolved magnesium carbonate preferably contains from about 50 mg to about 200 milligrams of magnesium per litre of water, more particularly from about 90 mg per litre to 150 mg per litre, even more particularly from about 100 mg per litre to about 140 mg per litre.

The aqueous solution comprising dissolved magnesium bicarbonate or dissolved magnesium bicarbonate and dissolved magnesium carbonate may have a concentration of bicarbonate anions from about 200 mg per litre to about 1000 mg per litre, more particularly from about 400 mg per litre to 800 mg per litre, even more particularly from about 500 mg per litre to 700 mg per litre.

The aqueous solution comprising dissolved magnesium bicarbonate or dissolved magnesium bicarbonate and dissolved magnesium carbonate preferably has a pH as high as possible within the range of from 7.5 to 8.9, consistent with the objective of maintaining a clear solution, without the higher pH causing magnesium carbonate containing species to be either precipitated from the solution or not being dissolved or converted into magnesium bicarbonate in the first place.

It has been found that a variable which influences the solubility of magnesium carbonate containing species is the relative concentrations of carbonate and bicarbonate in the solution, which, in turn, is a function of pH. Thus, the higher the pH, the greater the fraction that will be present in the form of carbonate, and the greater the likelihood, therefore, of magnesium carbonate precipitating or not going into solution.

The pH of the aqueous suspension of magnesium carbonate may advantageously be controlled to be above about 8, ideally between about 8.2 and about 8.6.

This may be achieved by either decreasing or increasing the pH of the aqueous suspension of magnesium carbonate and of the resulting solution by dissolving in the suspension a larger or a smaller amount of carbon dioxide.

Alternatively, the pH may be controlled by increasing or decreasing the amount of magnesium carbonate introduced into the suspension. As another alternative, the pH may be controlled by introducing into the solution a physiologically acceptable acid such as any acid acceptable for use in the food or beverage industries, including tartaric acid, citric acid, phosphoric acid or lactic acid.

The pH may be controlled by decreasing or increasing the pH of the aqueous suspension of magnesium carbonate by adding more or less of the species resulting from the dissolution of carbon dioxide in or by dissolving in the suspension a larger or a smaller amount of carbon dioxide. Alternatively, the pH may be controlled by increasing or decreasing the amount of magnesium carbonate introduced into the suspension. As another alternative, the pH may be controlled by introducing into the solution protons or a substance which has an effect on the pH.

As a source of magnesium cations, any compound of magnesium containing carbonate and/or hydroxide and/or oxide groups, whether or not in combination with other elements or groups of elements, may be used. Compounds of magnesium that may be used include the normal carbonates of magnesium, including magnesite [MgCO₃], magnesium oxide [MgO], barringtonite [MgCO₃.2H₂O], nesquehonite [MgCO₃.3H₂O] and lansfordite [MgCO₃.5H₂O], as well as the basic (or hydroxyl-containing) carbonates of magnesium having the general formula xMgCO₃.yMg(OH)₂.zH₂O, including artinite [MgCO₃.Mg(OH)₂.3H₂O], hydromagnesite [4MgCO₃.Mg(OH)₂.4H₂O], dypingite [4MgCO₃.Mg(OH)₂.5H₂O], and an as yet unnamed octahydrate [4MgCO₃.Mg(OH)₂.8H₂O]. It has been found that the pentahydrate or dypingite form of magnesium carbonate hydroxide works particularly well. It is also commercially available in food grade, which is required for the manufacture of potable solutions of magnesium bicarbonate that meets the strictest health standards.

Commercially, magnesium carbonate is available in two forms, one of which has a bulk density of about 0.4 g/mL whilst the other has a bulk density of about 0.2 to about 0.3 g/mL. The grade with the lower bulk density is preferred because it has been found to be more reactive and to dissolve at a faster rate in the process according to the invention, than the one with the higher bulk density.

It has been found that the degree to which magnesium carbonate is precipitated or prevented from dissolving or from reacting with the species resulting from the dissolution of carbon dioxide in water, is dependent on temperature, but also on the presence of other cations and anions in the aqueous solution, such as calcium ions which tend to combine with magnesium and iron cations in solutions as aforementioned to form various so-called double salts of magnesium carbonate such as huntite [CaMg₃(CO₃)₄], siderite [FeCO₃], dolomite [CaMg(CO₃)₂] or calcite [CaCO₃]. If desired, from a physiological perspective, to have calcium and iron cations present in the solution, then any of the aforementioned compounds may also be used as a source of magnesium cations.

The solubilities in water of some of the carbonates of magnesium, expressed as the solubility product Kₛₚ, are as follows:

| Compound | Solubility/Solubility Product |
|---|---|
| Magnesite [MgCO₃] | Solubility product*1: 1.0 × 10⁻⁵ |
| MgCO₃.H₂O | Solubility product*1: 2.7 × 10⁻⁵ |
| Barringtonite [MgCO₃.2H₂O] | Solubility product*1: 2.3 × 10⁻⁵ |
| Nesquehonite [MgCO₃.3H₂O] | Solubility product*1: 8.9 × 10⁻⁶ |
| Hydromagnesite | Solubility: .04 g per 100 cc in cold |
| [3MgCO₃.Mg(OH)₂.3H₂O] | water and .011 g per 100 cc in cold |
| | water*2 |
| Dypingite [4MgCO₃.Mg(OH)₂.5H₂O] | Solubility: 1 part in 300 parts |
| | water*3 |

*1Source: Kirk-Othmer: Encyclopedia of Chemical Technology, Fourth Edition, John Wiley and Sons, 1991, Vol 15, p680;
*2Handbook of Chemistry and Physics, 55^{th} Edition (1974 to 1975), B-105;
*3Merck Index

It is sometimes advantageous to prepare a magnesium bicarbonate solution containing calcium cations at a concentration at most equal to and preferably lower than that which would cause insoluble calcium species to either not dissolve or to be precipitated from the solution, thereby allowing the aqueous solution to clarify on standing. Calcium carbonate is less soluble than magnesium carbonate, with the result that calcium cations present in either the water or the magnesium carbonate to contribute to the formation of calcium carbonate (calcite) and/or dolomite and/or other insoluble calcium species, which contribute to cloudiness in the magnesium bicarbonate solution. Because of an equilibrium between carbonate and bicarbonate species in solution, which equilibrium is shifted in favour of carbonate species at increasing pH, the presence in the solution of calcium cations places a restriction on the amount of magnesium bicarbonate that can be maintained in solution within the desired pH range of 8.2 to 8.9. Accordingly, where it is important to maximise the amount of dissolved magnesium bicarbonate that is to be administered to a patient, the concentration of calcium cations in the water and in the magnesium carbonate that are used in the process according to the invention, is thus preferably as low as is practically possible.

In one embodiment of the invention, a source of magnesium carbonate is used which has been selected to have a low concentration of calcium cations. Preferably, the concentration of calcium cations in the source of magnesium carbonate is less than the concentration of calcium cations in the source of magnesium carbonate, for a given water quality, than a concentration of calcium cations that would correspond with the maximum solubility of calcium carbonate in a magnesium bicarbonate and magnesium carbonate solution in the said water, having a temperature falling within the temperature ranges and having a pH approaching the higher end of the pH ranges contemplated by the invention.

In another embodiment of the invention, a source of water is used which has been selected to have a low concentration of calcium cations. In this embodiment of the invention, the water may be selected so as to contain less calcium cations than would correspond with the maximum solubility of calcium carbonate in a magnesium bicarbonate and magnesium carbonate solution having a temperature falling within the temperature ranges and having a pH approaching the higher end of the pH ranges contemplated by the invention.

The invention also extends to a process for the manufacture of an aqueous solution comprising dissolved magnesium bicarbonate or dissolved magnesium bicarbonate and dissolved magnesium carbonate, including a step of reducing the concentration of calcium cations in the solution.

The concentration of calcium cations may be reduced by creating conditions favouring the precipitation of calcium carbonate and by the removal of precipitated calcium carbonate from the solution prior to bottling of the magnesium bicarbonate solution.

The solubility of calcium carbonate (calcite) is stated by the Handbook of Chemistry and Physics, 55^{th} Edition (1974 to 1975), page B-77 as 0.0014 grams per 100 cc in water at a temperature of 25° C. and 0.0018 grams per 100 cc in hot water (at a temperature of 75° C.). This equates to 14 and 18 mg per litre respectively. According to the same source, the solubility of magnesium carbonate (as the pentahydrate) is 0,176 g per 100 cc in cold water (1760 mg per litre), whilst the solubility of dolomite is 0,032 g per 100 cc in water at a temperature of 18° C. (32 mg per litre).

Thus, it is advantageous that the concentration of calcium compounds or ions in the water and any other ingredients used to prepare the aqueous solution is about equal to or lower than that whereby insoluble calcium species would result, thereby allowing a substantially clear aqueous solution to be prepared. The above solubility figures provide guidance as to the upper concentration levels of calcium ions that can be tolerated in the aqueous solution.

One way of removing calcium ions from the water used to prepared the aqueous solution, is by the distillation of the water.

Another way is by the use of reverse osmosis through a suitable membrane.

An alternative way to remove calcium ions is by means of ion exchange. Cation exchange may be done by replacing calcium ions with alkali metal cations or with protons.

Still another way is through the precipitation and removal, in a separate step, of calcium species. Thus, by taking advantage of the differences in solubilities of magnesium and calcium carbonates, by creating conditions in the solution which favour the presence of carbonate over the presence of bicarbonate and by accurately controlling the pH, it is possible to precipitate calcium carbonate in preference over magnesium carbonate.

One possibility is to pass the water through a a packed column containing magnesium carbonate.

Excess calcium ions may be precipitated from the solution. The precipitate may be separated from the solution by known methods of separation of solids from liquids, such as gravitational settling, filtration, and centrifugation.

The process according to the first aspect of the invention may also include the step of controlling the pressure at an absolute pressure of from about 50 kPa to about 500 kPa. In order to ensure or enhance the dissolution of carbon dioxide and/or to facilitate in the separation of calcium and magnesium, the partial pressure of carbon dioxide may be controlled within a desired range of 1×10⁻¹² atmospheres to 50 atmospheres, preferably above 1×10⁻¹⁰ atmospheres, more preferably, above 1×10⁻⁹ atmospheres.

The temperature of the aqueous suspension of magnesium carbonate may also be controlled. The temperature may be controlled between above about 0° C. and about 25° C. More specifically, the temperature may be controlled in the range between about 1° C. and about 20° C., more preferably below about 18° C., alternatively between about 2° C. and about 18° C., between about 2° C. and about 18° C., between about 2° C. and about 15° C., between about 2° C. and about 12° C., between about 2° C. and about 10° C., between about 2° C. and about 8° C., between about 2° C. and about 5° C., alternatively between about 5° C. and about 18° C., even more preferably in the range between about 10° C. and about 18° C., ideally in the range of from about 12° C. to about 18° C., even more ideally between about 12° C. and about 15° C., alternatively between about 5° C. and about 15° C., between about 5° C. and about 12° C., or between about 5° C. and about 10° C.

It is to be understood that at lower temperatures, the rate of dissolution of carbon dioxide in water is faster than at higher temperatures. However, the rate of the reaction of magnesium carbonate with the species resulting from the dissolution of carbon dioxide in water, to form magnesium bicarbonate, is lower at lower temperature. Thus, the temperature of the aqueous suspension of magnesium carbonate water may be controlled at such a level as to achieve a desired absorption of carbon dioxide into the suspension consistent with a desired rate of reaction between magnesium carbonate and the species formed as a result of the dissolution of carbon dioxide in the suspension. The solubility of carbon dioxide in water at various pressures and temperatures is as follows:

[image]

Source: Kirk-Othmer: Encyclopedia of Chemical Technology, Fourth Edition, John Wiley and Sons, 1991, Vol 5, p36

The contacting step of either the first or the second aspect of the invention may be conducted either continuously or batchwise.

By carefully following the teachings of the present invention, the aqueous solution of magnesium bicarbonate may be produced in commercial quantities in less than about 6 hours.

If required, the process in accordance with the first aspect of the invention may conveniently also include the additional step of bottling the said aqueous solution of magnesium bicarbonate or magnesium bicarbonate and magnesium carbonate, under an atmosphere containing carbon dioxide, preferably at an absolute pressure of from about 50 kPa to about 500 kPa. Alternatively, the solution may be bottled under air or nitrogen, at a pressure from about 50 kPa to about 500 kPa.

The bottling step is preferably carried out when the magnesium bicarbonate solution is clear, or, in order to reduce processing time, whilst the turbidity of the magnesium bicarbonate solution is no more than a slight haze and the pH falls within the range of about pH 7.0 to pH 9.0, ideally from about pH 7.8 to pH 8.8, more ideally from about pH 8.0 to pH 8.8. It has been found that if the turbidity is low enough, the solution will clear up after bottling if the pH falls within this range, but when the turbidity is too high, the solution either does not clear up after bottling or the end pH, after complete reaction of said species with the magnesium carbonate, is too high. If the pH value exceeds pH 9.0, the taste of the magnesium bicarbonate solution becomes unacceptable as it tends to be too caustic. Above pH 9.0 carbonate floccules may reappear in the solution. If the pH value falls below pH 7.0, the medical value of the magnesium bicarbonate solution is largely negated.

It has been found that by preparing an aqueous solution comprising dissolved magnesium bicarbonate or dissolved magnesium bicarbonate and dissolved magnesium carbonate that contains from about 120 mg per litre to about 140 mg per litre of magnesium ions, that has a pH of about 8.2 to about 8.8, that is substantially saturated with magnesium carbonate and contains so much magnesium bicarbonate as is consistent with the saturated concentration of magnesium carbonate, there is no need for the solution to be bottled with additional carbon dioxide, so that it can be bottled and handled as a “still” product.

In order to prevent the presence of unwanted foreign matter in the solution, the process according to the first aspect of the invention may include the step of passing any one or more of the suspension, the solution and the water to be used for the preparation of the aqueous magnesium carbonate suspension through a filter, conveniently having a maximum pore size of about 10 microns, preferably having a maximum pore size of about 5 microns, more preferably having a maximum pore size of about 2 microns.

If the magnesium bicarbonate solution according to the invention is to be used as a potable drink or for medical purposes, it should contain as few micro-organisms as possible. Ideally, there should not be any micro-organisms present in the solution.

The process according to the first aspect of the invention may thus include the additional step of sterilising at least one of and preferably all of the materials and any apparatus used in the manufacture of the solution comprising dissolved magnesium bicarbonate and magnesium carbonate. Sterilisation may be achieved by ozonation and/or by treatment with ultraviolet light.

Sterilisation of feed water or any of the process streams, suspensions or solutions is conveniently applied in steps. In a first step or series of steps, a source of feed water for use in the production of the solution comprising dissolved magnesium bicarbonate and magnesium carbonate may be treated with ozone so as to maintain a substantially constant ozone concentration of at least about 0.4 parts per million, preferably in the range of from about 0.3 to about 0.5 parts per million. In another step, the source of feed water for use in the production of the solution comprising dissolved magnesium bicarbonate and magnesium carbonate may be treated with ultra violet light. In additional or alternative steps, a suspension of the source of magnesium carbonate or a product solution of dissolved magnesium bicarbonate and magnesium carbonate may be treated with at least one of the ozone and ultraviolet treatments as described above.

The process according to the first aspect of the invention is preferably conducted in a clean environment, which may be managed in accordance with a ruling Clean In Place (CIP) code of practice.

Tests should be done regularly to ascertain whether the magnesium bicarbonate solution is sterile. As an example, a microbiological count should be done before and after UV light treatment and ozonation as described above.

According to a tenth aspect of the present invention, there is provided an apparatus for the manufacture of an aqueous solution comprising dissolved magnesium bicarbonate and dissolved magnesium carbonate, the apparatus comprising means for contacting an aqueous suspension of a source of magnesium cations with such an amount of a species resulting from the dissolution of carbon dioxide in water and having an initial pH below about 7 as is sufficient to result in the said aqueous solution comprising dissolved magnesium bicarbonate and dissolved magnesium carbonate having a pH of from about 7.5 to about 8.9 and to be substantially clear.

The apparatus according to the tenth aspect of the invention may conveniently include means for preparing the aqueous suspension of a source of magnesium cations. This means may also include means for keeping in suspension a major portion of said source of magnesium cations.

The apparatus according to the tenth aspect of the invention may further include means for preparing the aqueous solution comprising species resulting from the dissolution of carbon dioxide in water.

Conveniently, the apparatus according to the tenth aspect of the invention may include means for controlling the pH of the solution.

The apparatus according to the tenth aspect of the invention may also include means for controlling the pressure and/or the temperature of the solution.

The apparatus according to the tenth aspect of the invention may include means for bottling the said aqueous solution of magnesium bicarbonate and magnesium carbonate.

Filter means for removing from any one or more of the suspension, the solution and the water to be used for the preparation of the aqueous magnesium carbonate suspension, may also be provided. Conveniently, the filter has a maximum pore size of about 10 microns. The pore sizes are preferably a maximum of about 5 microns, more preferably a maximum of about 2 microns.

The apparatus according to the second aspect of the invention may further include means for sterilising the apparatus and at least one of and preferably all of the suspensions and solutions processed by the apparatus.

According to a eleventh aspect of the present invention, there is provided a method of preparing a substantially clear aqueous solution containing magnesium bicarbonate, including the steps of:
contacting, with species resulting from the dissolution of carbon dioxide in water, an aqueous suspension of magnesium carbonate, at suitable conditions of pressure and temperature, so as to obtain an aqueous solution of magnesium bicarbonate at a pH, after reaction of the said species with the magnesium carbonate, falling within a range of from about 8.0 to about 8.8; and
controlling the pH of the said solution within the said range until substantially all of the said magnesium carbonate has been converted to dissolved magnesium bicarbonate.

The pH may be controlled by decreasing or increasing the pH of the aqueous suspension of magnesium carbonate and of the resulting solution by dissolving in the suspension a larger or a smaller amount of carbon dioxide. Alternatively, the pH may be controlled by increasing or decreasing the amount of magnesium carbonate introduced into the suspension. As another alternative, the pH may be controlled by introducing into the solution protons or a substance which has an effect on the pH.

According to a twelfth aspect of the present invention, there is provided a method of preparing a substantially clear aqueous solution containing magnesium bicarbonate, including the steps of:
contacting, with species resulting from the dissolution of carbon dioxide in water, an aqueous suspension of magnesium carbonate, at suitable conditions of pressure and temperature, so as to obtain an aqueous solution of magnesium bicarbonate at a pH, after reaction of the said species with the magnesium carbonate, falling within a range of from about 8.0 to about 8.8; and
agitating the suspension so as to keep in suspension a major portion of the magnesium carbonate until substantially all of the said magnesium carbonate has been converted to dissolved magnesium bicarbonate.

The major portion of the magnesium carbonate may be any proportion from about 30% to about 100% of the magnesium carbonate forming part of the said suspension.

The method according to the twelfth aspect of the invention may conveniently also include a step in which the pH is controlled. This may be achieved by either decreasing or increasing the pH of the aqueous suspension of magnesium carbonate and of the resulting solution by dissolving in the suspension a larger or a smaller amount of carbon dioxide.

Alternatively, the pH may be controlled by increasing or decreasing the amount of magnesium carbonate introduced into the suspension. As another alternative, the pH may be controlled by introducing into the solution protons or a substance which has an effect on the pH.

The pH of the aqueous suspension of magnesium carbonate may advantageously be controlled to be above about 8, ideally between about 8.2 and about 8.6.

The method according to these aspects of the invention may include the step of controlling the pressure at an absolute pressure of from about 50 kPa to about 500 kPa.

The aqueous suspension of magnesium carbonate preferably contains from about 50 mg to about 200 milligrams of magnesium per litre of water, more particularly from about 90 mg per litre to 150 mg per litre, even more particularly from about 100 mg per litre to about 140 mg per litre. The aqueous solution of magnesium bicarbonate obtained may have a concentration of bicarbonate anions from about 200 mg per litre to about 1000 mg per litre, more particularly from about 400 mg per litre to 800 mg per litre, even more particularly from about 500 mg per litre to 700 mg per litre.

By carefully following the teachings of the present invention, the aqueous solution of magnesium bicarbonate may be produced in commercial quantities in less than about 6 hours.

The species resulting from the dissolution of carbon dioxide in water may include protons and bicarbonate ions. Alternatively or additionally, it may include carbonic acid.

The magnesium carbonate is preferably contacted with a stoichiometric quantity of species resulting from the dissolution of carbon dioxide in water, or with a quantity of such species which exceeds a stoichiometric quantity by from about 0% to about 20%, preferably by no more than about 10%, more preferably, by no more than about 1% to about 5%.

The temperature of the aqueous suspension of magnesium carbonate may also be controlled. The temperature may be controlled between about 0° C. and about 25° C.

It is to be understood that at lower temperatures, the rate of dissolution of carbon dioxide in water is faster than at higher temperatures. However, the rate of the reaction of magnesium carbonate with the species resulting from the dissolution of carbon dioxide in water, to form magnesium bicarbonate, is lower at lower temperature. Thus, the temperature of the aqueous suspension of magnesium carbonate water may be controlled at such a level as to achieve a desired absorption of carbon dioxide into the suspension consistent with a desired rate of reaction between magnesium carbonate and the species formed as a result of the dissolution of carbon dioxide in the suspension. Preferably, the temperature is maintained below about 25° C. in the range between about 1° C. and about 20° C., more preferably below about 16° C. in the range between about 5° C. and about 16° C., even more preferably in the range between about 10° C. and about 16° C., ideally in the range of from about 12° C. to about 16° C., even more ideally between about 12° C. and about 15° C.

The contacting step of either the eleventh or the twelfth aspect of the invention may be conducted either continuously or batchwise.

The method in accordance with the eleventh and twelfth aspects of the invention may conveniently also include the additional step of bottling the said aqueous solution of magnesium bicarbonate, preferably under an atmosphere containing carbon dioxide, preferably at an absolute pressure of from about 50 kPa to about 500 kPa. Alternatively, the solution may be bottled under air or nitrogen, at a pressure from about 50 kPa to about 500 kPa.

The bottling step is preferably carried out when the magnesium bicarbonate solution is clear, or, in order to reduce processing time, whilst the turbidity of the magnesium bicarbonate solution is no more than a slight haze and the pH falls within the range of about pH 7.0 to pH 9.0, usually at least pH 7.38, ideally from about pH 7.8 to pH 8.8 or pH 8.0 to 8.8, more ideally from about pH 8.0 to pH 8.6. It has been found that if the turbidity is low enough, the solution will clear up after bottling if the pH falls within this range, but when the turbidity is too high, the solution either does not clear up after bottling or the end pH, after complete reaction of said species with the magnesium carbonate, is too high. If the pH value exceeds pH 9.0, the taste of the magnesium bicarbonate solution becomes unacceptable as it tends to be too caustic. Above pH 9.0 carbonate floccules may reappear in the solution. If the pH value falls below pH 7.0, the medical value of the magnesium bicarbonate solution is largely negated.

In order to prevent the presence of unwanted foreign matter in the magnesium bicarbonate solution, the method according to the eleventh and twelfth aspects of the invention may include the step of passing any one or more of the suspension, the solution and the water to be used for the preparation of the aqueous magnesium carbonate suspension through a filter having a maximum pore size of about 10 microns, preferably having a maximum pore size of about 5 microns, more preferably having a maximum pore size of about 2 microns.

According to a thirteenth aspect of the present invention, there is provided an apparatus suitable for preparing an aqueous solution of magnesium bicarbonate, comprising:
means for contacting, with species resulting from the dissolution of carbon dioxide in water, a suspension of powdered magnesium carbonate in water so as to form an aqueous solution of magnesium bicarbonate; and
means for controlling the pH of the solution between about 7 and about 9, and, in particular between 7.8 and 9 or 8 and 8.8, by adjusting the amount of at least one of the said species and the said powdered magnesium carbonate that is contacted with the other.

The apparatus according to the thirteenth aspect of the invention may also comprise means for keeping in suspension a major portion of said powdered magnesium carbonate.

According to a fourteenth aspect of the present invention, there is provided an apparatus suitable for preparing an aqueous solution of magnesium bicarbonate, comprising:
means for contacting, with species resulting from the dissolution of carbon dioxide in water, a suspension of powdered magnesium carbonate in water so as to form an aqueous solution of magnesium bicarbonate; and
means for keeping in suspension a major portion of said powdered magnesium carbonate until substantially all of the suspended magnesium carbonate has been converted to dissolved magnesium bicarbonate.

The apparatus according to the fourteenth aspect of the invention may also comprise means for controlling the pH of the solution between about 7 and about 9, and, in particular between 7.8 and 9 or 8 and 8.8. This may be achieved by means adapted to adjust the amount of at least one of the said species and the said powdered magnesium carbonate that is present in the apparatus.

The apparatus according to the thirteenth and fourteenth aspects of the invention may also comprise means for bottling the solution of magnesium bicarbonate.

The means for contacting may comprise means, such as a tank of sufficient size, for containing the suspension and the species resulting from the dissolution of carbon dioxide in water. In a batch process, a tank large enough to contain all of the magnesium bicarbonate solution required for a production run in which a certain number of bottles are filled with the solution, may be provided. In the case of a continuous process, a smaller container may be sufficient for the contacting step, although a suitable reactor and/or clarifier or other similar but suitable pieces of equipment may be provided to ensure that the reaction between the magnesium carbonate and the said species is permitted to run to substantial completion.

The means for contacting with magnesium carbonate the species resulting from the dissolution of carbon dioxide in water may also comprise means for dissolving carbon dioxide in the water or in the said suspension so as to form a solution containing the said species.

The means for dissolving carbon dioxide may comprise a sparger comprising a fine mesh such as a stocking like mesh, or another suitable device capable of breaking up the carbon dioxide into small bubbles. The sparger may conveniently comprise a porous separation means adapted to separate pressurised carbon dioxide from water into which the carbon dioxide is to be dissolved, the pores of the separation means extending from a gas side thereof to a water side thereof, in use, and to break the carbon dioxide up into a plurality of small bubbles, for increasing the intimacy of contact between the carbon dioxide and the water.

The means for dissolving carbon dioxide may comprise, in addition to or as an alternative to the aforementioned sparger means, a tube and a helical baffle locatable within the tube, means for disposing the tube in a substantially vertical position, means for introducing carbon dioxide into a first end of the tube which, in use, is located below a second end of the tube, and means for introducing water or the said aqueous suspension of magnesium carbonate at either of the first end or the second end thereof, whereby carbon dioxide is allowed to bubble through the tube and to be intimately contacted with the water or said aqueous suspension of magnesium carbonate.

The apparatus may further comprise means for bottling the magnesium carbonate solution. The means may be or may comprise means for bottling the solution with air, nitrogen or carbon dioxide under a pressure from about 120 kPa to about 500 kPa.

According to a fifteenth aspect of the present invention, there is provided a method of preparing a substantially clear aqueous solution containing magnesium bicarbonate, including the steps of:
contacting, with species resulting from the dissolution of carbon dioxide in water, an aqueous suspension of magnesium carbonate, at suitable conditions of pressure and temperature, so as to obtain an aqueous solution of magnesium bicarbonate at a pH, after reaction of the said species with the magnesium carbonate, falling within a range of from about 8.0 to about 8.8;
controlling the pH of the said solution within the said range until substantially all of the said magnesium carbonate has been converted to dissolved magnesium bicarbonate; and
bottling the said magnesium bicarbonate solution.

According to a sixteenth aspect of the present invention, there is provided a method of preparing a substantially clear aqueous solution containing magnesium bicarbonate, including the steps of:
contacting, with species resulting from the dissolution of carbon dioxide in water, an aqueous suspension of magnesium carbonate, at suitable conditions of pressure and temperature, so as to obtain an aqueous solution of magnesium bicarbonate at a pH, after reaction of the said species with the magnesium carbonate, falling within a range of from about 8.0 to about 8.8;
agitating the suspension so as to keep in suspension a major portion of the magnesium carbonate until substantially all of the said magnesium carbonate has been converted to dissolved magnesium bicarbonate; and
bottling the said aqueous magnesium bicarbonate solution.

According to a seventeenth aspect of the present invention, there is provided an apparatus suitable for preparing an aqueous solution of magnesium bicarbonate, comprising:
means for contacting, with species resulting from the dissolution of carbon dioxide in water, a suspension of powdered magnesium carbonate in water so as to form an aqueous solution of magnesium bicarbonate;
means for controlling the pH of the solution between about 7 and about 9 by adjusting the amount of at least one of the said species and the said powdered magnesium carbonate that is contacted with the other; and
means for bottling the said solution of magnesium bicarbonate.

According to an eighteenth aspect of the present invention, there is provided an apparatus suitable for preparing an aqueous solution of magnesium bicarbonate, comprising:
means for contacting, with species resulting from the dissolution of carbon dioxide in water, a suspension of powdered magnesium carbonate in water so as to form an aqueous solution of magnesium bicarbonate;
means for keeping in suspension a major portion of said powdered magnesium carbonate until substantially all of the suspended magnesium carbonate has been converted to dissolved magnesium bicarbonate; and
means for bottling said solution of magnesium bicarbonate.

### BRIEF DESCRIPTION OF THE DRAWINGS

The present invention will now be described by way of example with reference to the accompanying drawings wherein:

FIG. 1 is a diagrammatical flow diagram of one portion of a process in accordance with the first aspect of the invention and an apparatus in accordance with the second aspect of the invention;

FIG. 2 is a diagrammatical flow diagram of another portion of a process in accordance with the first aspect of the invention and an apparatus in accordance with the second aspect of the invention;

FIG. 3 is a diagrammatic representation of an apparatus in accordance with the thirteenth and fourteenth aspects of the invention; and

FIG. 4 is a diagrammatic cross-sectional view of a sparger forming part of the apparatus of FIG. 3.

### DETAILED DESCRIPTION OF THE DRAWINGS

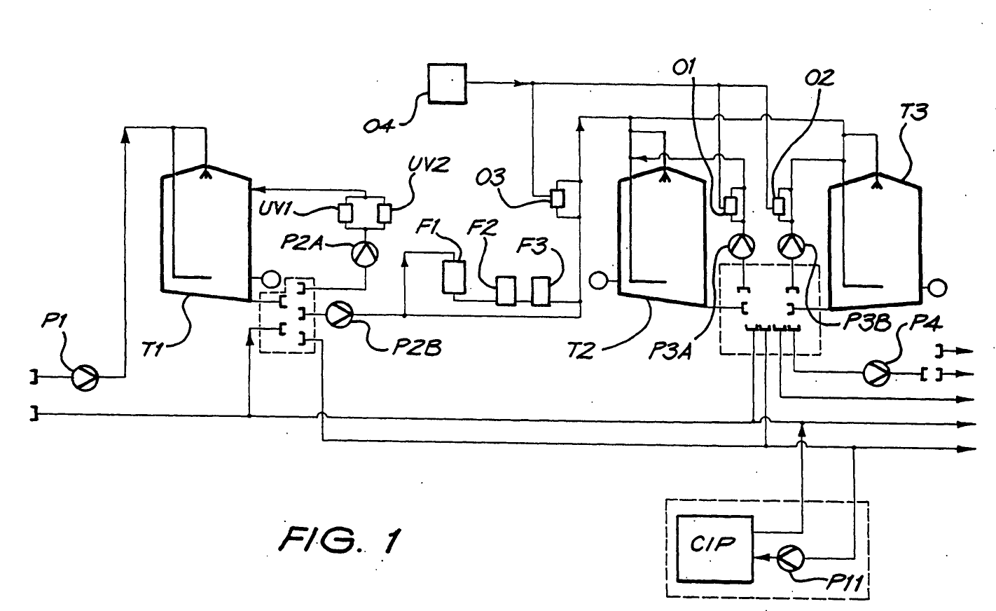

Referring to FIG. 1, there is shown a first portion of an apparatus 10 for the manufacture of an aqueous solution comprising dissolved magnesium bicarbonate.

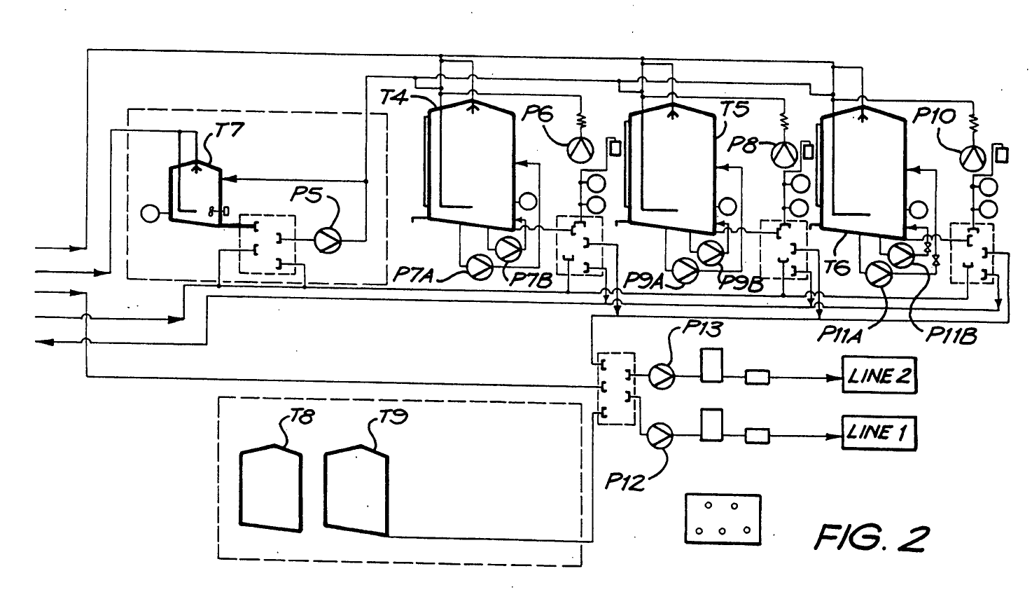

A second portion of the apparatus 10 is shown in FIG. 2.

The apparatus 10 comprises a tank T1 for receiving and for treating raw water by means of ultraviolet light. The raw water is transferred into the tank T1 by means of an unloading pump P1. A recirculating pump P2A is provided for recirculating the contents of tank T1 through either one or both of two ultraviolet treatment units UV1 and UV2.

From the tank T1, ultraviolet light treated water is transferred, by means of a transfer pump P2B, via a series of filters F1, F2 and F3, to one of two tanks T2 and T3 for ozone treatment. The filter F1 has a nominal pore size of 1 micron, whilst the filter F2 has a nominal pore size of 0.45 micron and the filter F3 has a nominal pore size of 0.2 micron.

After filtration, the water is passed through an ozone generator O1, in a single pass, so as to achieve an ozone concentration of around 0.2 ppm in the water, before it is discharged into either tank T2 or tank T3.

Whilst in the tanks T2 and T3, the water is recirculated through an ozone generator O2, using one of two recirculating pumps, respectively numbered P3a and P3b, until the ozone concentration reaches about 0.4 ppm.

Once an ozone concentration of about 0.4 ppm is reached, the now sterilised is transferred from the tanks T2 and T3 to one of the tanks T4, T5 and T6.

When a desired amount of sterilised water has been transferred to one of the tanks T4, T5 and T6, as may be determined by means of level measurements, a measured amount of precipitated magnesium carbonate powder is added to the sterilised water in the tank and mixed whilst the contents of the respective tank is being recirculated by means of one of three sets of circulating pumps P7A, P7B; P9A, P9B and P11A, P11B, one set being operatively connected to each of the tanks T4, T5 and T6. The two pumps of each set are used simultaneously to recirculate water of the respective tank, one of the pumps of these sets being operatively connected to a corresponding lower side entry nozzle located on the respective tank, whilst the other pump is operatively connected to a corresponding upper side entry nozzle located at a level about halfway up the side of the tank. The lower side entry nozzle is directed upwards, at an angle, whilst the upper side entry nozzle is directed downwards, at an angle. When the contents of the tank are circulated, the nozzles cause jets of water to be ejected into the contents of the tank, in a substantially vertical plane, rather than to swirl in a circular motion. It has been found that a circular motion is disadvantageous in that it facilitates separation of the solids from the suspension rather than to facilitate mixing. The lower nozzle may be directed towards the centre of the reactor tank, at an angle to the vertical and the upper side entry nozzle may be directed downwards, towards the centre of the reactor tank, at an angle to the vertical. By mixing in the manner described, particularly if the conditions in the tank are sufficiently turbulent, an intimate contact between the magnesium carbonate particles and the water can be achieved.

Whilst the contents of the respective tank T4, T5 or T6 is being mixed, a corresponding recirculating pump P6, P8 or P10 is used to contact the contents of the respective tank T4, T5 or T6 with carbon dioxide gas. The carbon dioxide gas is dissolved in the water on the suction side of the respective pump P6, P8 and P10, by means of a carbon dioxide injector, before the carbonated water is returned to the respective tank T4, T5, T6.

The pH of the solution containing species resulting from the dissolution of carbon dioxide is monitored, and the dissolution of carbon dioxide is proceeded with until the pH is no lower than about 8.2. It has been found that if the pH is taken down too far, it takes a long time for it to return to the ideal range of about 8.2 to about 8.6.

Once a substantially clear solution of magnesium carbonate and magnesium bi-carbonate has been produced in the tanks T4, T5 and T6, the contents is transferred to one of two bottling lines 1 and 2, by means of transfer and bottling pumps P12, P13.

Optional storage tanks T8, T9 are provided for storage of finished product.

Conveniently, the apparatus 10 includes means (not shown) for controlling the pH of the solution.

The apparatus 10 may also include means (not shown) for controlling the pressure and/or the temperature of the solution.

The apparatus 10 may further include means (not shown) for bottling the said aqueous solution of magnesium bicarbonate.

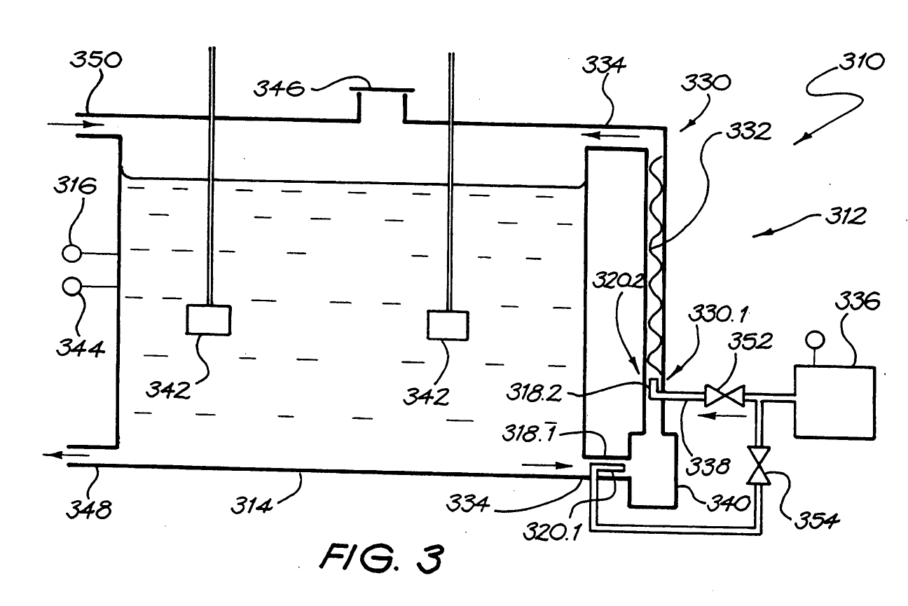

Referring to FIG. 3, there is shown an apparatus 310 suitable for preparing an aqueous solution of magnesium bicarbonate. The apparatus 310 comprises means 312 for contacting, with species resulting from the dissolution of carbon dioxide in water, a suspension of powdered magnesium carbonate in water so as to form an aqueous solution of magnesium bicarbonate.

The means 312 comprises a batch tank 314 of about 27 000 l capacity for containing the aqueous suspension of magnesium carbonate. In a batch process, the tank 314 is preferably large enough to contain sufficient suspension of magnesium carbonate for a desired production run. In the case of a continuous process, the tank 314 may be of a relatively smaller size, just enough for the contacting step to take place. However, a suitable reactor which may be in the form of a clarifier or other similar piece of equipment, may be provided downstream of the contacting step, to ensure that the reaction between the magnesium carbonate and the said species will run to substantial completion.

The apparatus 310 further comprises means 316 for measuring and controlling the pH of the solution between about 8.0 and about 8.6.

The means 312 for contacting the said species with magnesium carbonate also comprises means 318.1, 318.2 for dissolving carbon dioxide in water or in the said suspension so as to form a solution containing the said species.

The means 318.1, 318.2 for dissolving carbon dioxide comprises a sparger 320.1, 320.2 or other suitable device for breaking up the carbon dioxide into small bubbles. The sparger 320.1, 320.2 may discharge carbon dioxide into and may co-operate with a venturi or similar device located in a pipe through which water may be passed.

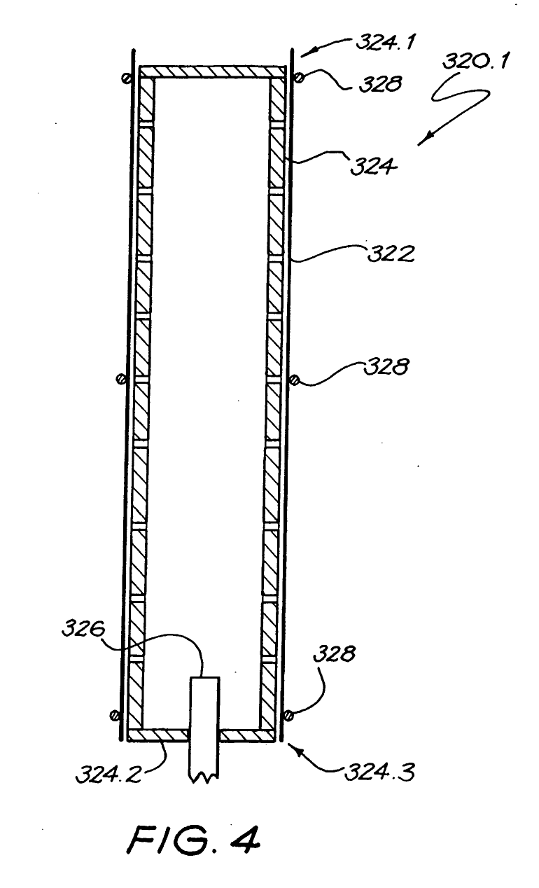

As is shown in FIG. 4, the sparger 320.1 comprises a porous separation means 322 (as is shown in FIG. 4, made of a suitable fabric or porous membrane, in the form of a woman's stocking, is disposed around a tube 324 having a plurality of holes drilled through its sides. The upper end 324.1 of the tube 324 is blanked off, whilst a further tube 326 of smaller cross-section conveys carbon dioxide into the tube 324 through a closure 324.3 covering its bottom end 324.3. The separation means 322 are held tight on the outside of the tube 324 by means of suitable rings or bands 328.

The separation means 322 is capable of separating pressurised carbon dioxide from the suspension or water into which the carbon dioxide is to be dissolved, the pores of the separation means 322 extending from a gas side thereof, which is located on the inside of the tube 324, to a water side thereof, in use, which is located on the outside of the separation means 322. The pores through the separation means are capable of breaking the carbon dioxide up into a plurality of small bubbles, so as to increase the intimacy of contact between the carbon dioxide and the water.

The means for dissolving carbon dioxide comprises, in addition to or as an alternative to the aforementioned sparger means 320.1, 320.2, a tube 330 and a helical baffle 332 locatable within the tube 330, and means 334 for disposing the tube 330 in a substantially vertical position.

The means for dissolving carbon dioxide further comprises means in the form of a carbon dioxide source 336 and a pipe 338 for introducing carbon dioxide into a first end 330.1 of the tube which, in use, is located below a second end 330.2 of the tube 330. The means for dissolving carbon dioxide also comprises means in the form of an external circulating pump 340 for introducing water or the said aqueous suspension of magnesium carbonate at the first end 330.1 of the tube 330, whereby carbon dioxide is allowed to bubble through the tube 330 and to be intimately contacted with the water or said aqueous suspension of magnesium carbonate.

The means 334 may be a pipe connected to the tank 314 and introducing liquid into the tank 314 through one or more distributors, which may be in the form of pipes with laterally extending holes through which the carbon dioxide enriched suspension or water may be injected into the tank 314 at positions strategically relative to the submersible pumps, so as to maximise the agitation and turbulence in the tank 314.

The means 16 for measuring and controlling may further comprise means (not shown) for respectively adding a caustic or an acidic solution to the contents of the tank 312 when the pH is either too low or too high, or else the control means may be adapted to or may comprise means adapted to reduce or increase the amount of carbon dioxide which is dissolved in the suspension. The means 16 may alternatively or additionally comprise an exhaust system to remove excess carbon dioxide, which tends to lower the pH value of the suspension and solution, from above the liquid inside the tank 314.

The apparatus may further comprise means for bottling the magnesium carbonate solution (not shown) with carbon dioxide under a pressure of from about 120 kPa to about 500 kPa.

Submersible pumps 342 are provided to further increase agitation in the tank 314. They may be replaced by suitable mixers. The submersible pumps and mixers are lowered and controlled inside the tank 314 so that the surface of the suspension or solution is not excessively mixed with the carbon dioxide blanket which forms in layers above the surface of the suspension and solution. This is particularly important when the reaction nears completion and as the volumes of the suspension and solution inside tank 314 decrease during the emptying or bottling processes.

Means 344 are provided for measuring and controlling the temperature of the water or suspension inside the tank 314. The temperature is conveniently controlled around 15° C.

Magnesium carbonate, which may conveniently be in the form of commercially available magnesium carbonate hydroxide pentahydrate (MgCO₃)₄.Mg(OH)₂.5H₂O is added to the tank 314 through an opening 346 in its top side. The magnesium carbonate may be in powder form or alternatively, it may be in the form of a slurry with water, or else in a concentrated suspension pre-mixed in a mixing tank (not shown).

An outlet 348 for magnesium bicarbonate solution and an inlet 350 for fresh water make-up, are also provided.

Valves 352, 354 are provided so as to be able to inject carbon dioxide either on the suction side of the pump 340 or on its discharge side, or both.

The pressure above the liquid surface in the tank 314 is preferably controlled at a level sufficiently high to ensure that carbon dioxide dissolves at a high enough rate for the reaction between magnesium carbonate and the said species to take place at a rate high enough to ensure that the processing time is reduced to a maximum of a few hours, preferably to a maximum of about 4 to 8 hours, more preferably, to a maximum of 1 to 2 hours. As was mentioned above, the amount of carbon dioxide taking part in the reaction may be controlled, particularly towards the end of the reaction, by removal of excess carbon dioxide by means of the aforementioned exhaust system.

Means (not shown) may be provided for measuring the turbidity of the solution in the tank 314, so as to determine when the solution is ready for bottling.

### EXAMPLE 1

### Preparation of Mildly Alkaline Magnesium Bicarbonate Aqueous Solution

A magnesium bicarbonate solution having a pH value of about pH 8.3 and containing about 125 mg of magnesium cations per litre and 650 mg of bicarbonate anions per litre was prepared in a 27,000 litre tank by reacting magnesium carbonate suspended as a powder in water with carbon dioxide dissolved in the water. The reaction time was 3 hours.

The pH of the contents of the tank was controlled so as to have an end value of 8.3. Initially the pH was higher than 8.3, even as high as about, but it was controlled by controlling the amounts of the magnesium carbonate powder that was added to the tank as well as the amount of carbon dioxide that was dissolved in the water.

The temperature of the contents of the tank was maintained at 15° C.

The contents of the tank were continuously agitated by means of two submersible pumps suspended in the tank, and by an external pump taking suction from the tank and discharging carbon dioxide enriched suspension back into the tank.

Carbon dioxide was dissolved in the water in the external circulation circuit, either on the suction side of the circulating pump, or, alternatively, on the discharge side, before subjecting the water and carbon dioxide mixture to a helically shaped baffle disposed inside the external tube of the circulation circuit. After reaction of the carbon dioxide with the magnesium carbonate and when the reaction was substantially or finally complete, i.e. when the turbidity was zero or no more than a slight haze, the specific aqueous solution of magnesium bicarbonate was passed through a 1 micron filter and bottled.

1. A method of preparing a substantially clear aqueous solution containing magnesium bicarbonate, including the steps of:
contacting, with species resulting from the dissolution of carbon dioxide in water, an aqueous suspension of magnesium carbonate, at suitable conditions of pressure and temperature, so as to obtain an aqueous solution of magnesium bicarbonate; and
controlling the pH of the said solution so that, after reaction of the said species with the magnesium carbonate, the final pH falls within a range of from about 8.0 to about 8.8,
wherein the concentration of calcium compounds or ions in the water and any other ingredients used to prepare the aqueous solution is about equal to or lower than that whereby insoluble calcium species would result, thereby allowing a substantially clear aqueous solution to be prepared.

2. A method of preparing a substantially clear aqueous solution containing magnesium bicarbonate, including the step of contacting, with species resulting from the dissolution of carbon dioxide in water, an aqueous suspension of magnesium carbonate, at suitable conditions of pressure and temperature, so as to obtain an aqueous solution of magnesium bicarbonate and magnesium carbonate, said aqueous solution having a pH, after reaction of the said species with the magnesium carbonate, within a range of from about 8.0 to about 8.8, wherein the concentration of calcium compounds or ions in the water and any other ingredients used to prepare the aqueous solution is about equal to or lower than that whereby insoluble calcium species would result, thereby allowing a substantially clear aqueous solution to be prepared.

3. The method of claim 2 further comprising controlling the pH of the suspension.

4. A method of preparing a substantially clear aqueous solution containing magnesium bicarbonate, including the steps of:
contacting, with species resulting from the dissolution of carbon dioxide in water, an aqueous suspension of magnesium carbonate, at suitable conditions of pressure and temperature, so as to obtain an aqueous solution of magnesium bicarbonate at a pH, after reaction of the said species with the magnesium carbonate, falling within a range of from about 8.0 to about 8.8; and
agitating the suspension as to keep in suspension a major portion of the magnesium carbonate until substantially all of the said magnesium carbonate has been converted to dissolved magnesium bicarbonate,
wherein the concentration of calcium compounds or ions in the water and any other ingredients used to prepare the aqueous solution is about equal to or lower than that whereby insoluble calcium species would result, thereby allowing a substantially clear aqueous solution to be prepared.

5. The method of claim 4 further comprising controlling the pH of the suspension.

6. The method of claim 4 further comprising controlling the temperature of the suspension between about 0° C. and about 25° C.

7. The method of claim 4 further comprising bottling the said aqueous solution of magnesium bicarbonate.

8. The method of claim 4 further comprising bottling the said aqueous solution of magnesium bicarbonate at a pH within a range of from about 7.8 to about 9.

9. The method of claim 4 further comprising bottling the said aqueous solution of magnesium bicarbonate when the magnesium bicarbonate solution is clear, or when the turbidity of the magnesium bicarbonate solution is no more than a slight haze and the pH falls within the range of about pH 7.8 to about pH 9.0.

10. An apparatus suitable for preparing an aqueous solution of magnesium bicarbonate, comprising:
means for contacting, with species resulting from the dissolution of carbon dioxide in water, a suspension of powdered magnesium carbonate in water so as to form an aqueous solution of magnesium bicarbonate; and
means for controlling the pH of the solution between about 7 and about 9 by adjusting the amount of at least one of the said species and the said powdered magnesium carbonate that is contacted with the other.

11. An apparatus suitable for preparing an aqueous solution of magnesium bicarbonate, comprising:
means for contacting, with species resulting from the dissolution of carbon dioxide in water, a suspension of powdered magnesium carbonate in water so as to form an aqueous solution of magnesium bicarbonate; and
means for keeping in suspension a major portion of said powdered magnesium carbonate until substantially all of the suspended magnesium carbonate has been converted to dissolved magnesium bicarbonate.

12. The apparatus according to claim 11 further comprising means for controlling the pH of the solution between about 7.8 and about 9.

13. The apparatus according to claim 111 further comprising means for bottling the solution of magnesium bicarbonate.

14. The apparatus according to claim 11 wherein the means for contacting further comprises means for dissolving carbon dioxide in the water or in the said suspension so as to form a solution containing the said species.

15. The apparatus according to 14 wherein the means for dissolving carbon dioxide comprises a sparger comprising a fine mesh such as a stocking like mesh.

16. The apparatus according to claim 15 wherein the means for dissolving carbon dioxide further comprises a tube and a helical baffle locatable within the tube, means for disposing the tube in a substantially vertical position, means for introducing carbon dioxide into a first end of the tube which, in use, is located below a second end of the tube, and means for introducing water or the said aqueous suspension of magnesium carbonate at either of the first end or the second end thereof, whereby carbon dioxide is allowed to bubble through the tube and to be intimately contacted with the water or said aqueous suspension of magnesium carbonate.

17. The apparatus of claim 11 further comprising means for bottling the magnesium bicarbonate solution.

18. A method of preparing a substantially clear aqueous solution containing magnesium bicarbonate, including the steps of:
contacting, with species resulting from the dissolution of carbon dioxide in water, an aqueous suspension of magnesium carbonate, at suitable conditions of pressure and temperature, so as to obtain an aqueous solution of magnesium bicarbonate at a pH, after reaction of the said species with the magnesium carbonate, falling within a range of from about 8.0 to about 8.8;
controlling the pH of the said solution within the said range until substantially all of the said magnesium carbonate has been converted to dissolved magnesium bicarbonate; and
bottling the same magnesium bicarbonate solution,
wherein the concentration of calcium compounds or ions in the water and any other ingredients used to prepare the aqueous solution is about equal to or lower than that whereby insoluble calcium species would result, thereby allowing a substantially clear aqueous solution to be prepared.

19. A method of preparing a substantially clear aqueous solution containing magnesium bicarbonate, including the steps of:
contacting, with species resulting from the dissolution of carbon dioxide in water, an aqueous suspension of magnesium carbonate, at suitable conditions of pressure and temperature, so as to obtain an aqueous solution of magnesium bicarbonate at a pH, after reaction of the said species with the magnesium carbonate, falling within a range of from about 8.0 to about 8.8;
agitating the suspension so as to keep in suspension a major portion of the magnesium carbonate until substantially all of the said magnesium carbonate has been converted to dissolved magnesium bicarbonate; and
bottling the said aqueous magnesium bicarbonate solution,
wherein the concentration of calcium compounds or ions in the water and any other ingredients used to prepare the aqueous solution is about equal to or lower than that whereby insoluble calcium species would result, thereby allowing a substantially clear aqueous solution to be prepared.

20. An apparatus suitable for preparing an aqueous solution of magnesium bicarbonate, comprising:
means for contacting, with species resulting from the dissolution of carbon dioxide in water, a suspension of powdered magnesium carbonate in water so as to form an aqueous solution of magnesium bicarbonate;
means for controlling the pH of the solution between about 7 and about 9 by adjusting the amount of at least one of the said species and the said powdered magnesium carbonate that is contracted with the other; and
means for bottling the said solution of magnesium bicarbonate.

21. An apparatus suitable for preparing an aqueous solution of magnesium bicarbonate, comprising:
means for contacting, with species resulting from the dissolution of carbon dioxide in water, a suspension of powdered magnesium carbonate in water so as to form an aqueous solution of magnesium bicarbonate;
means for keeping in suspension a major portion of said powdered magnesium carbonate until substantially all of the suspended magnesium carbonate has been converted to dissolved magnesium bicarbonate; and
means for bottling said solution of magnesium bicarbonate.

22. A method for the manufacture of an aqueous solution comprising dissolved magnesium bicarbonate and dissolved magnesium carbonate, wherein the process includes the step of contacting and aqueous suspension of a source of magnesium cations with such an amount of a species resulting from the dissolution of carbon dioxide in water and having an initial pH below about 7 as is sufficient to result in the said aqueous solution comprising dissolved magnesium bicarbonate and dissolved magnesium carbonate having a pH of from about 8 to about 8.9 and to be substantially clear, wherein the concentration of calcium compounds or ions in the water and any other ingredients used to prepare the aqueous solution is about equal to or lower than that whereby insoluble calcium species would result, thereby allowing a substantially clear aqueous solution to be prepared.

23. A method for the manufacture of an aqueous solution comprising dissolved magnesium bicarbonate or dissolved magnesium bicarbonate and dissolved magnesium carbonate, wherein the process includes the step of: (i) contacting an aqueous suspension of a source of magnesium cations with bubbles of carbon dioxide gas, (ii) agitating the contacted aqueous suspension, (iii) recontacting the agitated contacted aqueous suspension with bubbles of carbon dioxide gas, and (iv) repeating steps (i) to (iii) as required, to provide a pH in the aqueous solution of from about 8 to about 9, wherein the concentration of calcium compounds or ions in the water and any other ingredients used to prepare the aqueous solution is about equal to or lower than that whereby insoluble calcium species would result, thereby allowing a substantially clear aqueous solution to be prepared.

24. The method according to claim 1 further including sterilizing the water and the suspension.

25. The method according to any one of claim 1, wherein the concentration of calcium compounds or ions in the water is reduced by creating conditions favouring the precipitation of calcium carbonate and by the removal of such precipitated calcium carbonate from the solution.

26. An apparatus for the manufacture of an aqueous solution comprising dissolved magnesium bicarbonate and dissolved magnesium carbonate, the apparatus comprising means for contacting an aqueous suspension of a source of magnesium cations with such an amount of a species resulting from the dissolution of carbon dioxide in water and having an initial pH below about 7 as is sufficient to result in the said aqueous solution comprising
dissolved magnesium bicarbonate and dissolved magnesium carbonate having a pH of from about 7.5 to about 8.9 and to be substantially clear.
